(19) United States
(12) Patent Application Publication
(10) Pub. No.: US 2022/0054163 A1
(43) Pub. Date: Feb. 24, 2022

(54) IMPLANTABLE NERVE BLOCKING INTERVENTION

(71) Applicant: John Mansell, Gillette, WY (US)

(72) Inventor: John Mansell, Gillette, WY (US)

(21) Appl. No.: 17/275,627

(22) PCT Filed: Sep. 13, 2019
(86) PCT No.: PCT/US2019/051026

§ 371 Date: Mar. 11, 2021

Related U.S. Application Data
(60) Provisional application No. 62/731,591, filed on Sep. 14, 2018.

(51) Int. Cl. A61B 17/34

(52) U.S. Cl. CPC A61B 17/3415; A61B 2017/348; A61B 17/3423; A61B 17/3401

(57) ABSTRACT

The implantable nerve blocking intervention involves performing a first subdermal incision on the ventral lateral portion of the patient's thorax. A first end of a tunneling device is inserted through the first subdermal incision. The tunneling device is traversed below the patient's dermal layer towards the mid axillary line. The tunneling device is traversed above the lower section of the rib cage towards the patient's paralumbar line. A longitudinal paramidline incision is performed about 3 inches lateral to the vertebral body and the tunneling device. A needle or trocar tip is inserted through the paramidline incision towards a primitive cluster of nerves and a catheter is placed through the needle or trocar which is removed prior to anchoring the catheter. A catheter tube is inserted through the length of the tunneling device. The catheter tube is coupled to the distal catheter. The tunneling device is retracted over the catheter tube. The catheter tube is coupled to a percutaneous access port.

### CROSS-REFERENCE TO RELATED APPLICATIONS

This application claims priority to U.S. Provisional Patent Application No. 62/731,591 filed on Sep. 14, 2018 and entitled “Implantable Nerve Blocking Intervention.”

### BACKGROUND

Chronic pain syndromes, such as complex regional pain syndrome, chronic pelvic pain and chronic abdominal pain caused by such diseases as acute and chronic pancreatitis, are difficult to treat through conventional pain management interventions. These syndromes often require complicated interventions where a needle is utilized to inject an anesthetic to block activity of the autonomic nerves responsible for sensing the visceral pain. These interventions are inherently risky due to the proximity of vascular structures, viscera as well as to the spinal cord and its supplying blood vessels and exiting nerve roots near the neural structures associated with these syndromes. Incorrect needle placement can damage viscera, the spinal cord, lungs, or other neural structures. In some instances it may lead to infection of intervertebral disks, the central nervous system, or the abdominal cavity.

In order to reduce the risk of potential complications, these interventions typically involve the use of expensive fluoroscopy and require an experienced pain specialist well versed in the regional anatomy and experienced in the interventional pain management techniques to operate. They expose the doctor and patient to radiation. With the lifelong nature of some of these syndromes the cumulative risk of repeated radiation exposure is significant. The cumulative expense over the lifetime of the patient is significant. The lack of immediate access to this procedure leads to frequent emergency department visits and increases the risk of opiate addiction. While these interventions are effective, they are not long lasting in their palliative duration and repeated injections increase the risk of complications even with an experienced pain specialist. Therefore, a need exists for an accessible pain management intervention for chronic pain syndromes.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an implanted subdermal catheter device 100 in accordance with one embodiment.

FIG. 2 illustrates a subdermal catheter device 200 in accordance with one embodiment.

FIG. 3 illustrates a tunneling device 300 in accordance with one embodiment.

FIG. 4 illustrates a method 400 for installing an implantable nerve block intervention device in a patient in accordance with one embodiment.

FIG. 5 illustrates an anterior view 500 of a patient during the implant procedure in accordance with one embodiment.

FIG. 6 illustrates a lateral view 600 of a patient during the implant procedure in accordance with one embodiment.

FIG. 7 illustrates a posterior view 700 of a patient during the implant procedure in accordance with one embodiment.

FIG. 8 illustrates a posterior view 800 of a patient during the implant procedure in accordance with one embodiment.

FIG. 9 illustrates a lateral view 900 of a patient during the implant procedure in accordance with one embodiment.

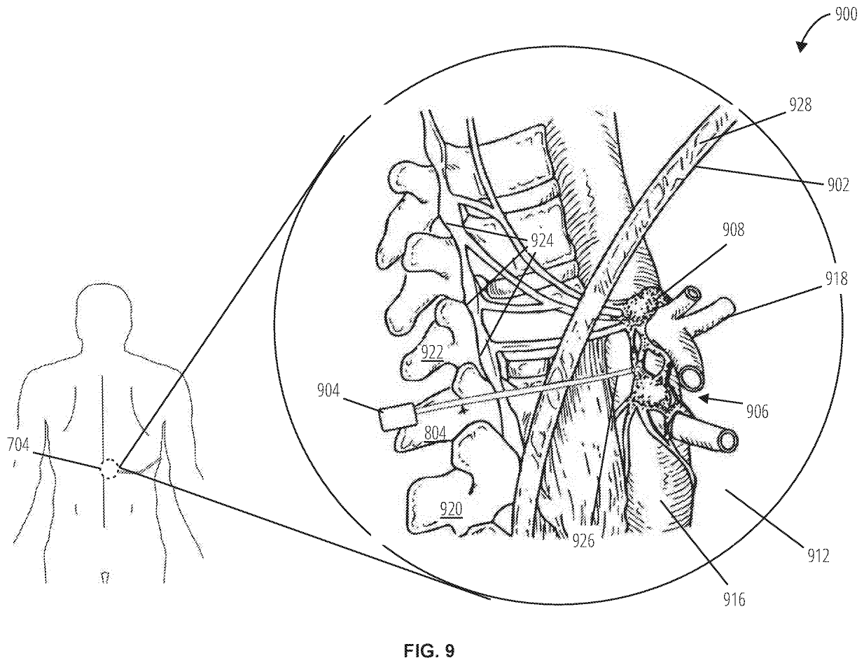

FIG. 10 illustrates a cross sectional view 1000 of a patient at L1 during the implant procedure in accordance with one embodiment.

FIG. 11 illustrates a lateral view 1100 of a patient during the implant procedure in accordance with one embodiment.

FIG. 12 illustrates a cross sectional view 1200 of a patient at L1 during the implant procedure in accordance with one embodiment.

FIG. 13 illustrates a cross sectional view 1300 of the patient during a dye test in accordance with one embodiment.

FIG. 14 illustrates a cross sectional view of a two-part tunneling device 1400 in accordance with one embodiment.

FIG. 15 illustrates a lateral view 1500 of a patient during the implant procedure in accordance with one embodiment.

FIG. 16 illustrates a method 1600 for installing an implantable nerve block intervention device utilizing a two-part tunneling device in a patient in accordance with one embodiment.

### DETAILED DESCRIPTION

An implantable nerve blocking intervention catheter procedure installs a subdermal catheter device for delivering an anesthetic to an autonomic or visceral nerve structure anterior to the vertebral body of L1 or L5, anterolateral to the lumbar vertebral bodies from L2 to L4 near the lumbar sympathetic chain or in the thoracic paravertebral space near the thoracic spinal nerves which innervate the trunk and parietal peritoneum. These nerve structures include the celiac plexus near L1, the superior hypogastric plexus near L5, the lumbar sympathetic chain at multiple lumbar levels and thoracic spinal nerves, the origin of the intercostal nerves. The subdermal catheter device includes a percutaneous access port coupled to a catheter positioned underneath the skin of the patient, and terminating at the anchored distal catheter tip in the prevertebral space or the anterolateral aspect of the vertebral body of L1, L5, the thoracic paravertebral space or near the sympathetic chain at the appropriate level. An anesthetic, such as bupivacaine may then be injected through the percutaneous access port and flow through the length of the catheter tube exiting through the terminus of the catheter, coating and thereby anesthetizing the celiac plexus, the superior hypogastric plexus, the thoracic spinal nerves after exiting the neuraxis or the lumbar sympathetic nerves.

The implantable nerve catheter involves performing a first subdermal incision on a first side of the ventral lateral portion of the patient's thorax between a patient's collar bone and nipple, where the port is to be placed. Based on patient anatomy and preference the port may be placed in other locations and some patients may prefer placement near the costal margin over the ribs or even below the costal margin over the abdominal wall. In addition to being the pocket for the subdermal port it is also the starting point for tunneling to the paraspinous incision, a tunnel which may be made in one or two segments depending on the size and anatomy of a particular patient. After developing a pocket for the subdermal port access, a first end of a tunneling device, comprising a conduit, is then inserted through this first subdermal incision. The first end of the tunneling device is then traversed below the patient's dermal layer towards the mid axillary line of the patient on the first side, where the tunneling device path is along the lateral trunk staying caudad (or “low) enough to avoid certain nerves such as the long thoracic nerve. If possible, the tunnel is continued around posteriorly to the paraspinous incision in a single operative step. If not, then another subdermal incision may be made to develop a second tunnel that is a continuation of the first segment in order to reach the paraspinous incision subdermally. Prior to developing the subdermal tunnel from the anterior trunk subdermal port to the back, a longitudinal incision is made parallel to the midline of the spine at the desired level of the distal catheter tip placement far enough from the midline to allow oblique catheter place in a prevertebral or paravertebral location. This incision should include dissection beneath the skin for placement of an anchoring device as well as to develop a tension loop that helps to avoid placing traction on the implanted distal catheter tip. Based on preoperative imaging of the targeted nerves to assess depth of needle placement, a graduated needle or trocar is placed to the desired depth, not exceeding and preferably less than the image-estimated distance to critical structures like the aorta, vena cava or similarly avoidable anatomy. This needle gauge is large enough to allow passage of the similarly-graduated catheter to the tip of the needle which is then removed while fixing the catheter in place so as to remain in place. A catheter is then inserted in the subdermal port proximal end of the tunnel device conduit, through the length of the tunneling device. The tunneled catheter may reach the paraspinous incision in one or two segments depending upon the size and anatomy of the patient. After injecting radiocontrast under live fluoroscopy through the prevertebral or paravertebral placed distal catheter placement to confirm satisfactory spread of the injectate, the distal catheter segment is connected to the proximal segment after removing the needle or trocar that was tunneled from the subdermal injection port via a fluid communicable coupling after the tunneling device is retracted over the catheter tube through the first (and possibly second) subdermal incision. Prior to connecting to the distal segment a non-crimping anchor is used to secure the distal segment in its prevertebral or paravertebral location. The proximal end of the catheter tube is then coupled to the percutaneous access port with a fluid communicable connector.

The implantable nerve blocking intervention catheter will then require a radiocontrast dye test to check injecting through the subdermal port for inappropriate resistance to injection, fluoroscopic evidence of leaks at any of the intervening connections, and again satisfactory spread of the contrast at the injection target of the pre- or paravertebral space. Once the dye test is satisfied, all of the incisions are closed over the subdermal port, the paraspinous incision and any potential intervening tunneling incision.

The implantable nerve blocking catheter intervention may be performed to treat chronic abdominal pain associated with the function of the celiac plexus. In this configuration, the medial lateral incision is performed adjacent to the first lumbar vertebral body (L1). The catheter tip is then inserted through the lateral paraspinous incision towards the celiac plexus in the prevertebral space of the L1. Success in treating loin pain hematuria syndrome with visceral nerve blocks has been noted. For patients with parietal peritoneal irritation a similar catheter may be used for paravertebral placement.

The implantable nerve blocking intervention may be performed to treat chronic pelvic syndrome associated with the activity of the superior hypogastric plexus. In this configuration, the lateral paraspinous incision is performed adjacent to the fifth lumbar vertebral body (L5). The catheter tip is then inserted through the paraspinous incision towards the superior hypogastric plexus in the prevertebral aspect of L5. There are an estimated 5 million women in the United States suffering from chronic pelvic pain.

The implantable nerve blocking intervention may be performed to treat complex regional pain syndrome associated with the activity of the lumbar sympathetic chain. In this configuration, the lateral paraspinous incision is performed adjacent to the vertebral body of the second, third or fourth vertebral bodies (L2, L3 or L4). The catheter tip is then inserted through the paraspinous incision towards the lumbar sympathetic chain in the paravertebral aspect of these three levels, L2, L3 or L4.

The celiac plexus is the largest of the three sympathetic plexuses of the autonomic nervous system. It is situated at the level of the first lumbar vertebra behind the stomach and in front of the crura of the diaphragm and the beginning of the abdominal aorta. The celiac plexus is composed of the two large ganglia, the celiac ganglia, and the dense network of nerve fibers connecting them together the plexus and the ganglia receive splanchnic nerves from both sides and some filaments from the vagus nerve. Numerous secondary plexuses branch from the celiac plexus, including the renal plexuses innervating the kidneys, the suprarenal plexus innervating, the suprarenal glands, the splenic and the hepatic plexuses innervating the spleen and the liver, the gastric plexus and the vagus branches innervating the stomach, the superior and the inferior mesentric plexuses innervating the intestines, and the testicular ovarian plexuses innervating, respectively, the testes in males and ovaries in the females. Blockade of the celiac plexus has been effective in reducing pain in any of the structures.

The sympathetic innervation of the abdominal viscera originates in the anterolateral horn of the spinal cord. Preganglionic fibers from the T5-T12 exit the spinal cord in conjunction with the ventral roots to join the white communicating rami on their way to the sympathetic chain. Rather than synapsing with the sympathetic chai, these preganglionic fibers pass through it to ultimately synapse on the celiac ganglia. The greater, lesser, and least splanchnic nerves provide the major preganglionic contribution to the celiac plexus. The greater splanchnic nerve has its origin from the T5-T10 spinal roots. The nerve travels along the thoracic prevertebral border through the crus of the diaphragm into the abdominal cavity, ending on the celiac ganglion of its respective side. The lesser splanchnic nerve arises from the T10-T11 roots and passes with the greater nerve to the end at the celiac ganglion. The least splanchnic nerve arises from the T11-T12 spinal roots and passes through the diaphragm to the celiac ganglion.

Interpatient anatomical variability of the celiac ganglia is significant but the following generalization can be drawn from anatomical studies of the celiac ganglia. The number of ganglia varies from one to five, and they range in diameter from 0.5 to 4.5 cm. The ganglia lie anterior and anterolateral to the aorta. The ganglia located on the left are uniformly more inferior to the right sided counterparts by as much as a vertebral level, but both groups of ganglia lie below the level of the celiac artery. The ganglia usually lie about at the level of the first lumber vertebra.

Postganglionic fibers radiate from the celiac ganglia to follow the course of the blood vessel to innervate the abdominal viscera. These organs include much of the distal esophagus, stomach, duodenum, small intestine, ascending and proximal traverse colon, adrenal glands, pancreas, spleen, liver, and biliary system. It is these postganglionic fibers, the fibers arising from the preganglionic splanchnic nerves and the celiac ganglion that make up the celiac plexus. The diaphragm separates the thorax from the abdominal cavity while still permitting the passage of the thoracoabdominal structures, including the aorta, vena cava, and the splanchnic nerves. The diaphragmatic crura are bilateral structures that arise from the anterolateral surface of the upper two or three lumbar vertebrae and disks. The crura of the diaphragm serves as a barrier to effectively separate the splanchnic nerves from the celiac ganglia and the plexus below.

The celiac plexus is anterior to the crus of the diaphragm. The plexus extends in front of and around the aorta, with greatest concentration of the fibers anterior to the aorta.

The lumbar sympathetic chain is associated with complex regional pain syndrome associated with sympathetically mediated pain of the kidneys, ureters, genitalia, and lower extremities. Included in this category are phantom limb pain, reflex sympathetic dystrophy, causalgia, and a variety of peripheral neuropathies. Treatment of the lumbar sympathetic chain may assist in the palliation of pain secondary to vascular insufficiencies of the lower extremities, including pain secondary to frostbite, atherosclerosis, Beurger's disease, and arteritis secondary to collagen vascular disease and to maximize blood flow after vascular procedures on the lower extremities.

The preganglionic fibers of the lumbar sympathetic exit the intervertebral foramina along with the lumbar paravertebral nerves. After exiting the intervertebral foramen, the lumbar paravertebral nerve gives off a recurrent branch that loops back through the foramen to provide innervation to the spinal ligaments, meninges, and its respective vertebra. The upper lumbar paravertebral nerve also interfaces with the lumber sympathetic chain via the myelinated preganglionic fibers of the white rami communicantes. All five of the lumbar nerves interface with the unmyelinated post ganglionic fibers of the gray rami communicantes. At the level of the lumbar sympathetic ganglia, preganglionic, and postganglionic fibers synapse. Additionally, some of the postganglionic fibers return to their respective somatic nerves via the gray rami communicantes. Other lumbar sympathetic postganglionic fibers travel to the aortic and hypogastric plexus and course up and down the sympathetic trunk to terminate in distant ganglia.

In many patients, the first and second lumbar ganglia are fused. These ganglia and the remainder of the lumber chain and ganglia lie at the anterolateral margin of the lumbar vertebral bodies. The peritoneal cavity lies lateral and anterior to the lumbar sympathetic chain. Given the proximity of the lumbar somatic nerves to the lumbar sympathetic chain, the potential exists for both neural pathways to be blocked when performing blockade of the lumbar sympathetic ganglion.

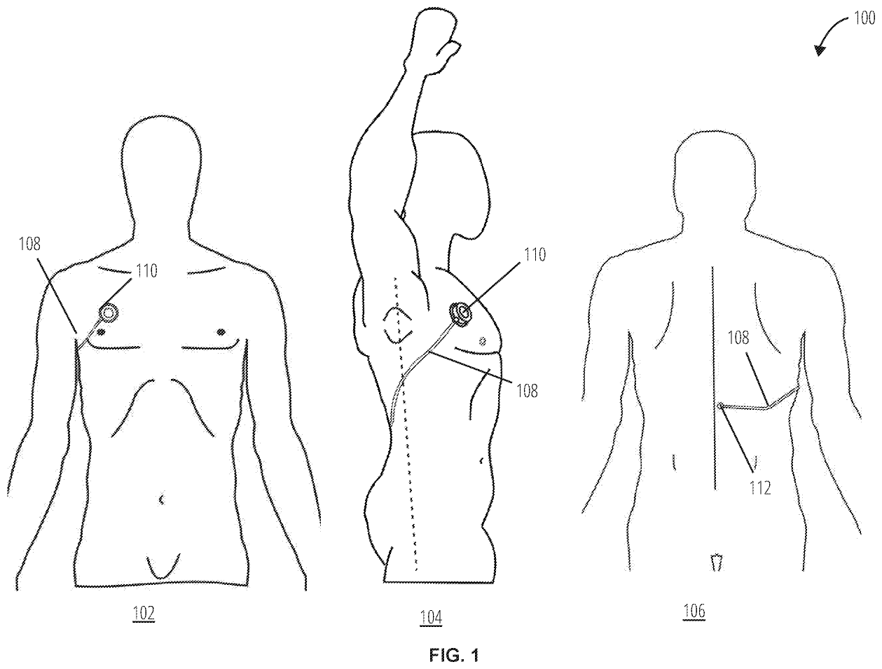

Referencing FIG. 1, an implanted subdermal catheter device 100 is shown in a patient in an anterior view 102, lateral view 104, and posterior view 106. The anterior view 102 shows the location of the percutaneous access port 110 installed on their upper chest with the subdermal catheter 108 angled underneath the armpit. The lateral view 104 shows the continuing path of subdermal catheter 108 underneath the patient's armpit and across the mid axillary line. The posterior view 106 shows the continuing path of the subdermal catheter 108 ending at the location of the implanted distal catheter 112 adjacent to the lateral aspect of a vertebral body.

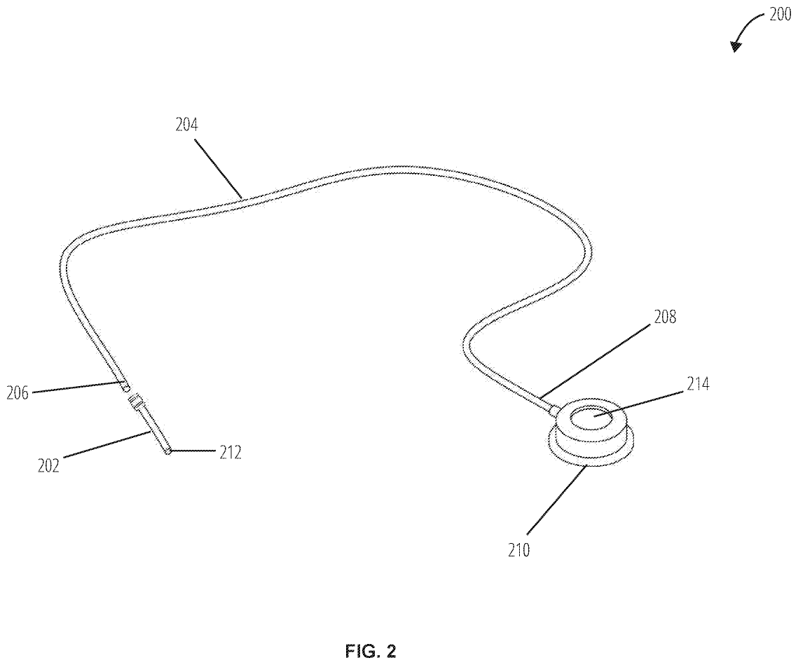

Referencing FIG. 2, a subdermal catheter device 200 is displayed in a partially assembled state outside of a patient's body. The subdermal catheter device 200 comprises a distal catheter tip 202, a catheter tube 204, and a percutaneous access port 210. The catheter tube 204 may be constructed of semi-rigid materials (e.g., silicone rubber, nylon, polyurethane, polyethylene terephthalate (PET), latex, thermoplastic elastomers, etc.) that are flexible enough to conform to the curvatures of a patient's body, but with enough rigidity to with stand external pressures that may cause the tube to buckle or kink underneath of the skin preventing the flow of anesthetic to the distal catheter tip 202. The percutaneous access port 210 is a medical appliance that is installed underneath the patient's skin and provides a site for injecting compounds into a patient. The percutaneous access port 210 comprises septum 214 through which a non-coring or Huber needle may be inserted to inject a compound. The septum 214 of the percutaneous access port 210 is in fluid communication with the catheter tube 204 by way of the proximal end 208 of the catheter tube 204, such that the compound injected through the septum 214 flows into and through the catheter tube 204 towards the distal catheter tip 202. The distal catheter tip 202 connects to the first end 206 of the catheter tube 204 and directs an injected compound towards a primitive cluster of nerves through the catheter tip 212. The catheter tip 212 includes an aperture where the injected compound exits from the subdermal catheter device 200.

The subdermal catheter device 200 may be utilized to deliver a local anesthetic to block a primitive cluster of nerves, such as the celiac plexus, the superior hypogastric plexus or the lumbar sympathetic ganglia of the lumbar sympathetic chain. In order to block the primitive cluster of nerves, 30 to 50 mL of bupivacaine may be injected through the septum of the percutaneous access port 210 and delivered to coat and anesthetize the nerve cluster.

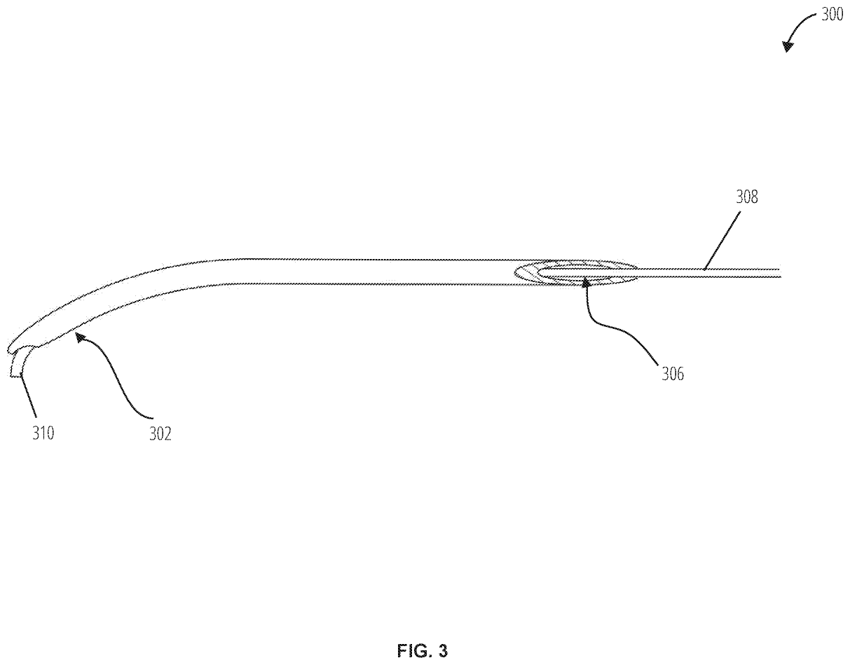

Referencing FIG. 3, a partial cross-sectional view of a tunneling device 300 is displayed comprising a first end 302 and a conduit 306. The conduit 306 traverses the length of the tunneling device 300 with the interior dimensions of the conduit being slightly larger than that of a catheter tube 308. The dimensions conduit 306 are provided relative to the catheter tube 308 in order to allow the catheter tube 308 to be threaded through the tunneling device 300. The first end 302 of the tunneling device 300 may include a curvature to facilitate movement when guided under the dermal layer of a patient. The first end 302 may include an aperture that opens up to the conduit 306 allowing the first end 310 of the catheter tube 308 to pass through.

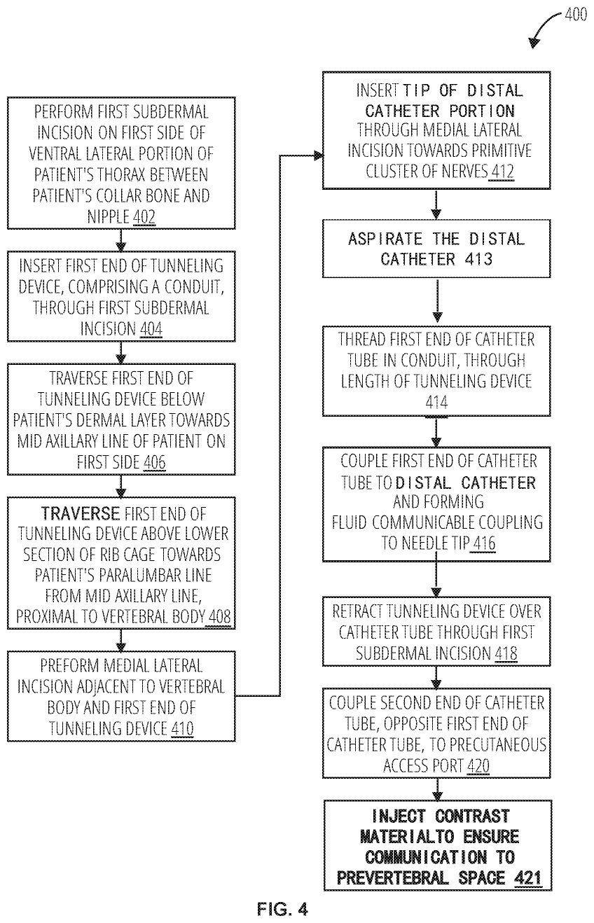

Referencing FIG. 4, a method 400 for installing an implantable nerve block intervention device in a patient involves performing a first subdermal incision on a first side of the ventral lateral portion of the patient's thorax between a patient's collar bone and nipple, where the first subdermal incision is found superior lateral to the nipple (block 402) In some embodiments of step 402, the proximal end of the device, the access port, may be placed between the collar bone and the nipple, over the lower anterior chest wall in the approximate mid-clavicular line, over the abdominal wall itself, although this would require a larger paddle underneath the port to support it during access. In block 404 of method 400, a first end of a tunneling device, comprising a conduit, is inserted through the first subdermal incision. In block 406 of method 400, the first end of the tunneling device traverses below the patient's dermal layer towards the mid axillary line of the patient on the first side, where the tunneling device path is well below the patient's armpit. In block 408 of method 400, the first end of the tunneling device traverses above the lower section of the rib cage towards the patient's paraspinous incision from the mid axillary line, adjacent to the desired vertebral body level. In some embodiments of steps 406 and 408, one or two tunnels may be required. For example, one tunnel may extend all the way from the anterior trunk to the posterior para-midline upper lumbar region, or two tunnels may be connected somewhere along the way by a joining connector due to the patient's body habitus and the inability to complete the tunnel in a single pass. In block 410 of method 400, a lateral paraspinous incision is performed adjacent to the vertebral body and the distal end of the tunneling device. In block 412 of method 400, a distal catheter portion is inserted large enough in its internal caliber to allow passage of the catheter to its targeted nerve structures. In block 413, the distal catheter may be aspirated for blood or spinal fluid before injecting contrast through the distal catheter to insure a pre-vertebral spread of the contrast prior to anchoring it with a non-crimping anchor into the deep dorsal fascia or musculature. In block 414 of method 400, the proximal end of a catheter tube is threaded in the conduit, through the length of the tunneling device. In block 416 of method 400, the distal end of the catheter tube is coupled to the proximal catheter segment forming a fluid communicable coupling to the distal catheter tip. In block 418 of method 400, the tunneling device is retracted over the catheter tube through the first subdermal incision. In block 420 of method 400, a second end of the catheter tube, opposite the first end of the catheter tube, is coupled to a percutaneous access port. In block 421, the method 400 may include injection of contrast material under live fluoroscopy through the proximal port to insure communication into the prevertebral space without obstruction or leakage.

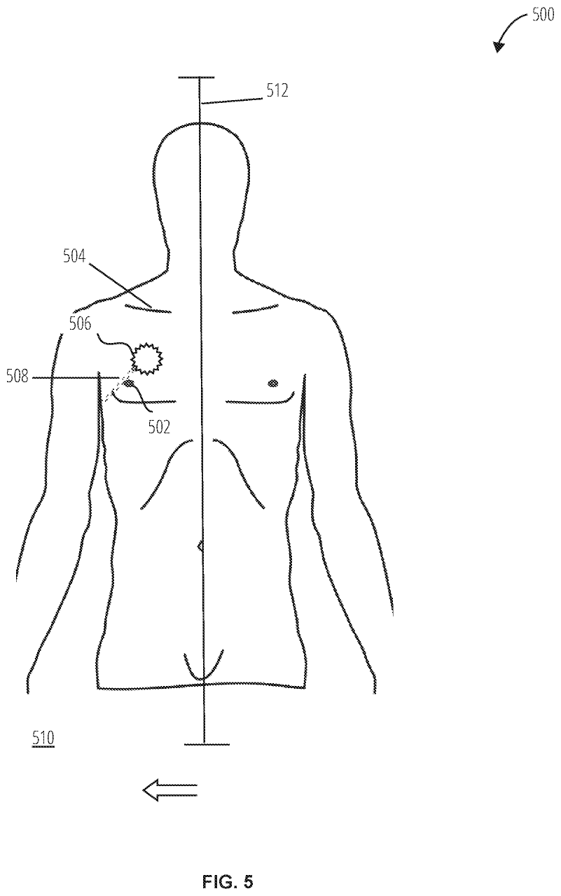

Referencing FIG. 5, an anterior view 500 of a patient is shown during the implant procedure. The anterior view 500 shows the location of the first subdermal incision 506 and the tunneling device path 508 for the tunneling device and subsequently the catheter tube. Alternate locations of the proximal access port may include over the lower rib cage or the right upper or middle quadrants. The first subdermal incision 506 is positioned ventral laterally on the first side 510, relative to the medial axis 512, of a patient's thorax between the collar bone 504 and the nipple 502. The first subdermal incision 506 is positioned superior lateral to the nipple but may be in other locations. When a tunneling device is inserted through the first subdermal incision 506, it is angled such that the tunneling device path 508 runs 4-5″ below the contents of the patient's arm pit as it is moves towards the mid axillary line. When tunneling device is removed and replaced by the catheter tube in the tunneling device path 508, the first subdermal incision 506 becomes the implant location for the percutaneous access port. The first side 510 may be right side of the patient's body

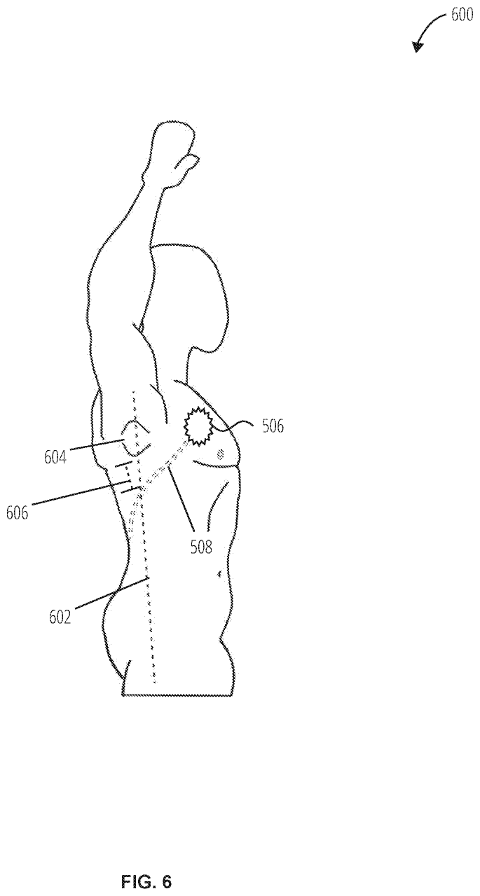

Referencing FIG. 6, a lateral view 600 of a patient is shown during the implant procedure. The lateral view 600 shows the location of the tunneling device path 508 as it traverses across the mid axillary line 602 from the first subdermal incision 506, towards the vertebral lateral aspect adjacent to the paralumbar line. The tunneling device path 508 is positioned below the patient's armpit 604 as it traverses across the mid axillary line 602. The distance 606 the tunneling device path 508 is positioned below the armpit 604 is about 4-5″ inches below the contents of the armpit 604 (e.g., axillary lymph nodes, etc.). From the mid axillary line 602, the tunneling device path 508 curves downward towards the bottom of the rib cage as it moves towards the vertebral lateral aspect of L1, L5 or other targeted levels.

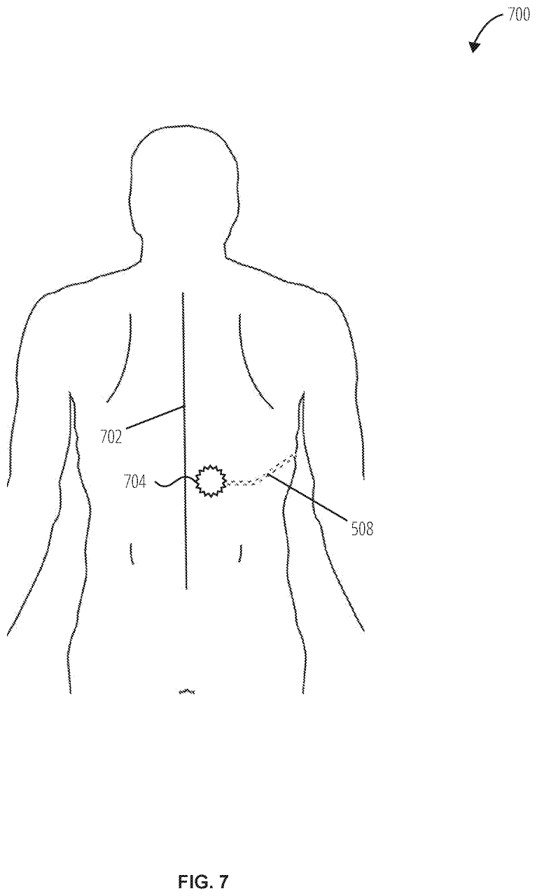

Referencing FIG. 7, a posterior view 700 of a patient is shown during the implant procedure. The posterior view 700 shows the continuation of the tunneling device path 508 from the mid axillary line 602 towards the paraspinous line 702 adjacent to the vertebral body of L1. When the tunneling device reaches the position lateral to the vertebral body of L1, a lateral paraspinous incision 704 has already been performed. The paraspinous incision 704 is the interim target during the procedure for the distal end of the tunneling device to where it is threaded. Following successful implantation of the distal catheter or catheter segment at the primitive cluster of nerves, the distal catheter segment is coupled, opposite the distal catheter tip, to the first end of the catheter tube, forming a fluid sealed and communicable coupling. The coupling between the proximal end of the catheter tube and the distal catheter segment, allows for the flow of a fluid compound towards to the space immediately adjacent to the targeted nerve structures. After the distal segment is anchored, the coupling is checked and a test injection from the subdermal port injection is performed to test the entire system, the paraspinous lumbar incision is typically closed.

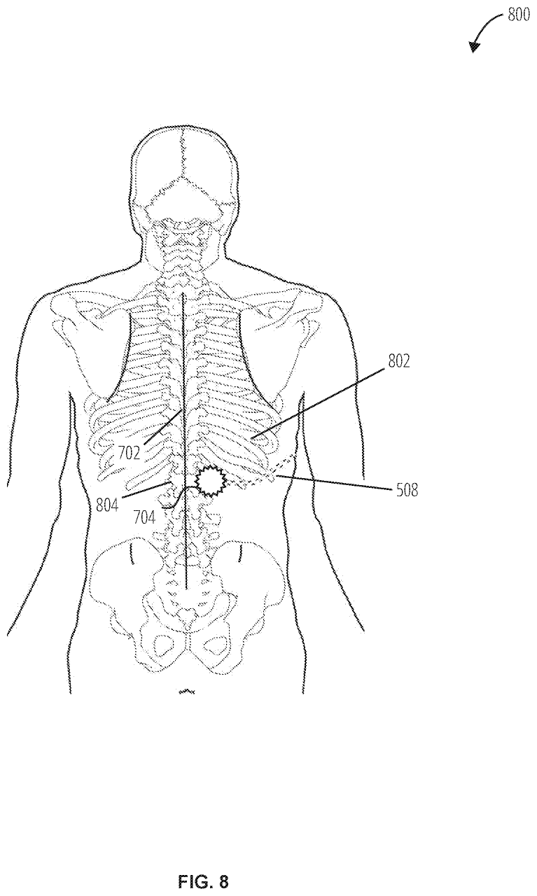

Referencing FIG. 8, a posterior view 800 of a patient is showing the skeletal structures underneath the patient's skin. The posterior view 800 shows the traversal of the tunneling device path 508 curving towards the bottom of the rib cage 802 towards the parapsinous line 702 adjacent to the vertebral body of L1 804. The paraspinous incision 704 is performed adjacent to the vertebral body L1 to L5, depending on the targeted nerve structures 804.

Referencing FIG. 8, lateral view 900 is an enhanced view of the distal catheter tip 926 of the distal catheter 904 positioned in the prevertebral space 912 of L1 804 adjacent to the celiac plexus 906. The vertebral body of L1 804 is found between T12 922 and L2 920. The distal catheter tip 926 traverses into the prevertebral space 912 adjacent to the vertebral body of L1 804 across the parietal peritoneum 902. The celiac plexus 906 comprises celiac ganglia 908 which are positioned near the celiac artery 918 on the aorta 916. Anterior to the prevertebral space 912 is the anterolateral aspect of the vertebral bodies where the lumbar sympathetic chain 924 is found on the margins of the vertebral body.

The positioning of the distal catheter tip via its disposable placing needle or trocar is similar to the positioning seen in a single needle transcrural/periaotic celiac plexus block intervention. The single needle transcrural/periaotic celiac plexus block uses local anesthetic as an indicator for diagnostic maneuvers to determine whether flank, retroperitoneal, or upper abdominal pain is sympathetically mediated via the celiac plexus. A regular interval transcrural celiac plexus block with local anesthetic also is useful in the palliation of pain secondary to acute pancreatitis and other acute pain syndromes mediated by the innervation of the celiac plexus. Early implementation of the celiac plexus block with local anesthetic, steroid, or both markedly reduces the morbidity and mortality associated with acute pancreatitis. Single needle periaortic celiac plexus block also is used to palliate the acute pain of arterial embolization of the liver from cancer therapy as well as to treat pain of abdominal “angina” associated with visceral arterial insufficiency. Single needle periaortic celiac plexus block with local anesthetic also may be used prognostically before celiac plexus neurolysis.

Pre-block preparations include the administration of adequate amounts of oral or intravenous fluids to attenuate the hypotension associated celiac plexus block. Evaluation of the patient for coagulopathy is indicated if the patient has undergone anti-neoplastic therapy or has a history of significant alcohol abuse with diminished liver function or damage. If radiographic contrast is to be used, evaluation of the patient's renal status should be considered.

The patient is placed in the prone position with a pillow placed under the abdomen to flex the thoracolumbar spine. For comfort, the patient's head is turned to the side or rested in a face cradle pillow, and the arms are permitted to hang freely off each side of the table, placed above the head or tucked by their sides. The inferior margins of the 12th ribs are identified and traced to the T12 vertebral body. The spinous process of the L1 vertebral body is then identified and marked with sterile marker. A point about 2.5 inches just inferior and lateral to each side of the transverse process of the L1 is identified. The injection sites are then prepared with antiseptic solution.

The skin, subcutaneous tissues, and the musculature are infiltrated with 1.0% lidocaine at the points of the needle entry. An appropriately sized needle is inserted unilaterally or bilaterally through the previously anesthetized area. The needle is initially oriented 45 degrees towards the midline and about 15 degrees cephalad to ensure contact with the L1 vertebral body. Once bone is contacted and the depth is noted, the needle is withdrawn to the level of the subcutaneous tissue and redirected slightly less mesiad (about 60 degrees from the midline) so as to walk off the lateral surface of the L1 vertebral body. The needle is reinserted to the depth at which the vertebral body was first contacted. At this point, if no bone is contacted the needle is gradually advanced 3 to 4 cm, or until the pulsation emanating from the aorta and transmitted to the advancing needle is noted. If aortic pulsations are noted, a pain specialist may either convert the block into a transaortic celiac plexus technique or note the depth to which the needle has been placed, withdraw the needle into the subcutaneous tissue, and then redirect the needle less mesiad to slide the laterally to the aorta. Ultimately, the tip of the needle should be in peri-aortic space. This periaortic precrural placement decreases the incidence of the inadvertent spread of injected solutions onto the lumbar somatic nerve root. The stylet of the needle is then removed, and the needle hub is inspected and aspirated for the presence of blood, cerebrospinal fluid, or urine.

For diagnostic and prognostic block via the single needle periaortic technique, 15 to 30 mL of a shorter or fast onset local anesthetic may be administered through the needles. For therapeutic block, 15 to 30 ml of 0.5% bupivacaine may be administered through the needle. Because of the potential for local anesthetic toxicity, all local anesthetic should be administered in incremental doses.

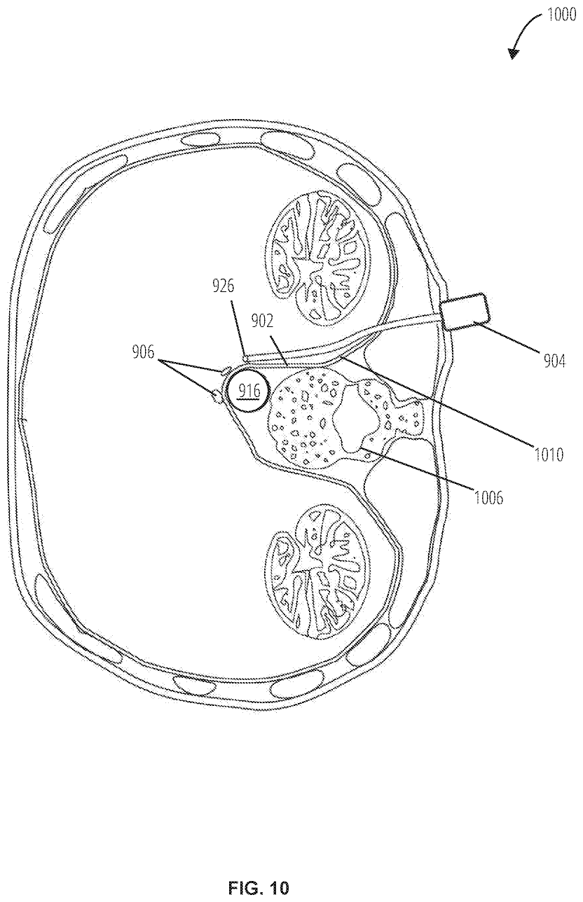

Referencing FIG. 10, a cross sectional view 1000 of a patient at L1 during the implant procedure is shown with a needle or trocar placement for a celiac plexus block. The distal catheter tip 926 of the distal catheter 904 is in the prevertebral space of L1 1006 adjacent to the celiac plexus 906, anterior to the aorta 916 though it may also be posterior. The distal catheter 904 placement is similar to the placement utilized in a single needle transcrural/periaortic celiac plexus block intervention, such that the distal catheter tip 926 can be seen traversing the diaphragm crura 1010 and into the parietal peritoneum 902 to reach the celiac plexus 906. With single needle transcural/periaortic approach to celiac plexus block, the needle is placed close to this concentration of plexus fibers. The relationship of the celiac plexus to the surrounding structure is as follows: The aorta lies anterior and slightly to the left of the anterior margin of the vertebral body. The inferior vena cava lies to the right, with the kidneys posterolateral to the great vessels. The pancreas lies anterior to the celiac plexus. All of these structures lie within the retroperitoneal space.

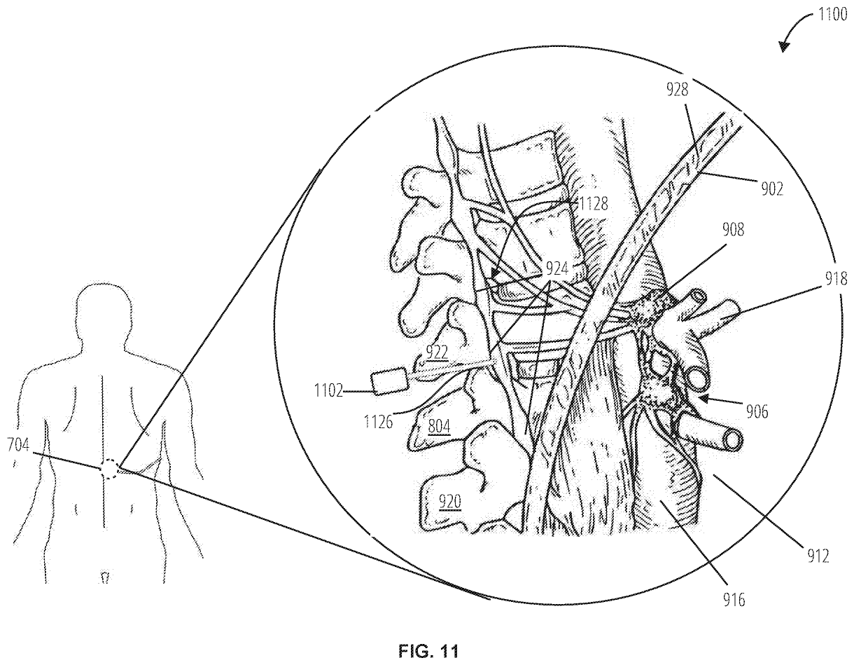

Referencing FIG. 11, a lateral view 1100 of a patient during the implant procedure shows an enhanced view of the longitudinal paramidline lateral incision 704 with the distal catheter tip 1126 of the distal catheter 1102 positioned within the anterior lateral aspect 1128 of the vertebral body of L1 804 with the distal catheter tip 1126 disposed towards the lumbar sympathetic chain 924. The incision will be located to facilitate a tunnel vision view of the targeted anatomy several centimeters lateral to the spinal midline. The anterior lateral aspect 1128 is found anterior to the prevertebral space 912 across the parietal peritoneum 902 and the crura of the diaphragm 928.

The positioning of the distal catheter 1102 is similar to the positioning of a needle utilized in a lumbar sympathetic ganglia block intervention. When performing a lumbar-sympathetic ganglia block a patient is placed in the prone position with a pillow under the abdomen to the gently flex the lumbar spine. The spinous process of the vertebrae just above the nerve to be blocked is palpated. At a point just below and 3 inches lateral to the spinous process, the skin is prepared with antiseptic solution. An appropriately sized needle (20 to 25 gauge from 3.5 to 6 inches in length) is attached to the 12-ml syringe and is advanced at a 35- to 45-degree angle to the skin, aiming for the lateral aspect of the vertebral body. The needle should impinge on bone after being advanced about 2 inches. If the needle comes into contact with bone at a shallower depth, it has probably impinged on the transverse process. If this occurs, the needle should be directed in a slightly more cephalad trajectory to pass above the transverse process to impinge on the lateral aspect of the vertebral body or just pass over its cortex under live fluoroscopy. After bony contact is made with the vertebral body, the needle is withdrawn into the subcutaneous tissues and redirected at a slightly steeper angle and walked off the lateral margin of the vertebral body. As soon as body contact is lost, the needle is slowly advanced about 0.5 inches deeper. Give the proximity of the lumbar sympathetic chain to the somatic nerve, a paresthesia in the distribution of the corresponding lumbar paravertebral nerve may be elicited if this occurs, the needle should be withdrawn and redirected slightly more cephalad.

The needle is then again slowly advanced until it passes the lateral border of the vertebral body. The needle should ultimately rest at the anterior lateral margin of the vertebral body. In fluoroscopy is used, a small amount of contrast medium may be added to the local anesthetic. The contrast medium should appear anterior to the vertebral body on the posteroanterior view and just lateral to the vertebral body on the lateral view. If computed tomographic guidance is used, the contrast can be seen surrounding the sympathetic chain anterolateral to the vertebral body. Once the needle is in position and careful aspiration reveals no blood or cerebrospinal fluid, 12 to 15 ml of 1.0% preservative free lidocaine is injected.

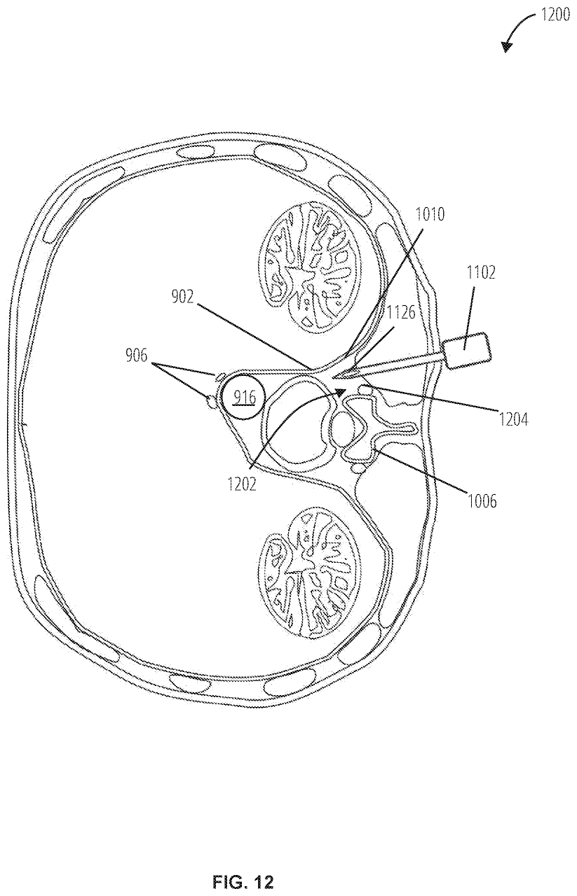

Referencing FIG. 12, the cross-sectional view 1200 of a patient at L1 during the implant procedure is shown with a needle or trocar placement for catheter insertion for a lumbar sympathetic chain block. The catheter tip 1126 of the needle 1102 is shown in the anterolateral aspect of L1 1202 adjacent to a lumbar-sympathetic ganglia 1204 of the lumbar sympathetic chain. The placement of the distal catheter 1102 and catheter tip 1126 is similar to the placement of the needle used in a lumbar sympathetic ganglia block intervention.

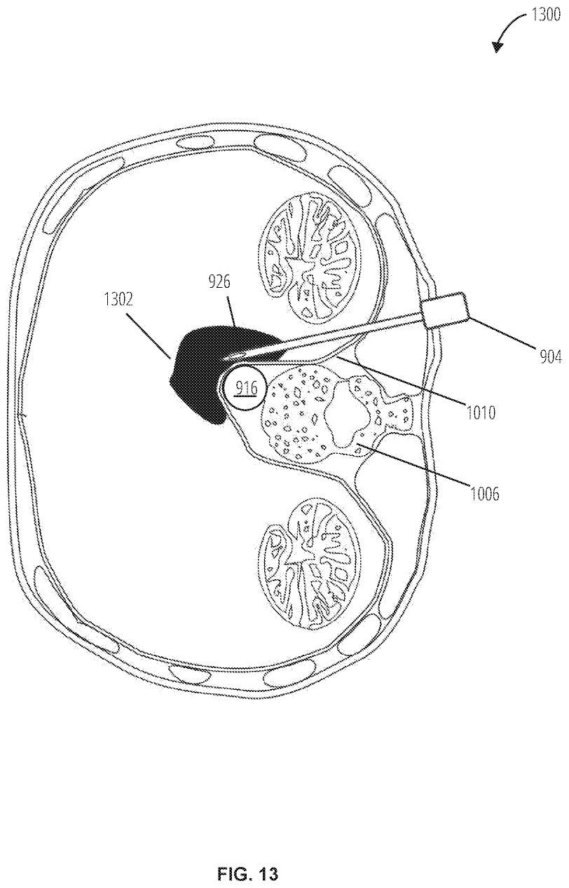

Referencing FIG. 13, a cross sectional view 1300 of the patient is shown at the vertebral body of L1 with the distal catheter tip 926 of the distal catheter 904 positioned to block the celiac plexus. The cross-sectional view 1300 shows the dispersion of a contrast material 1302 as it would appear under a CT scan. In practice, the contrast material 1302 is a fluorescent dye that would be viewed when using radiographic assistance or fluoroscopic assistance for implanting the catheter 904 using, anterior-posterior, lateral and lateral oblique views.

If radiographic guidance is being used, a small amount of contrast material is injected through the needle, and its spread is observed radiographically. On fluoroscopic anteroposterior view, contrast is confined primarily to the left of the midline near the L1 vertebral body. A smooth curvilinear shadow may be observed that corresponds to the contrast in the periaortic space of the lateral view. Alternatively, if computed tomographic guidance is used, contrast should appear periaortic or, if adenopathy or tumor is present, contrast should be confined to the periaortic space to the left of the aorta, or prevertebral. If this limitation of the spread contrast occurs, one should consider redirecting the catheter to facilitate better spread of the contrast around the aorta.

When utilized with the implanted nerve blocking device, the contrast material 1302 tests for kinks or deformations in the catheter tube. The lack of contrast material 1302 may be utilized to detect is there is a kink or deformation of the catheter tube.

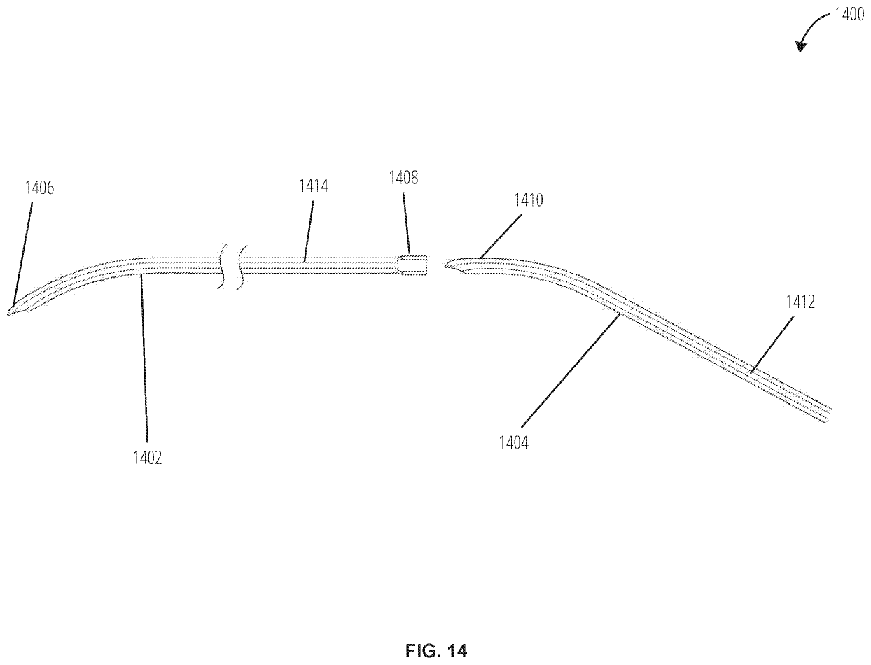

Referencing FIG. 14, a two-part tunneling device 1400 for installing an implanted nerve blocking device is shown in a cross-sectional view, although a tunneling device may be only one part. The two-part tunneling device 1400 comprises a second tunneling device 1402 and a first tunneling device 1404. The second tunneling device 1402 comprises a first end 1406, a second end 1408, and a conduit 1414 traversing the length of the second tunneling device 1402. The first tunneling device 1404 comprises a first end 1410 and a conduit 1412. The second end 1408 of the second tunneling device 1402 includes a widened portion adapted to optionally accept the first end 1410 of the first tunneling device 1404 though the first tunneling device could be used for both the anterior and posterior tunnels. The fitment of the first end 1410 within the second end 1408 allows for alignment of the conduits of the second tunneling device 1402 and the first tunneling device 1404.

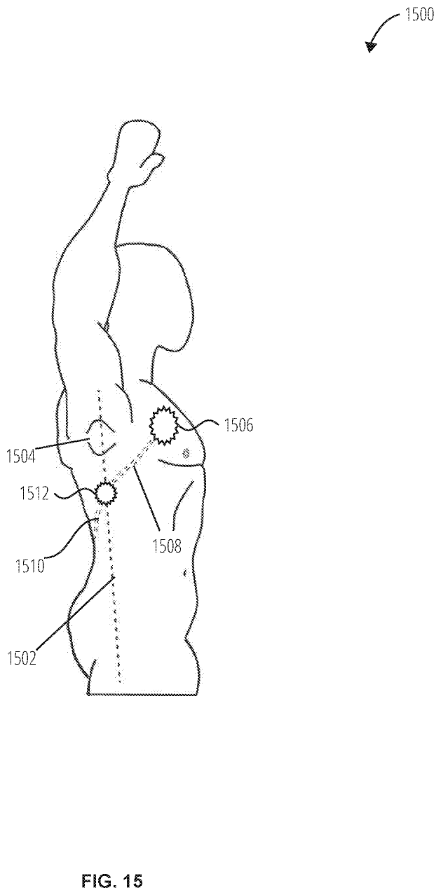

Referencing FIG. 15, the lateral view 1500 a patient during the implant procedure shows the location of a second subdermal incision 1512 utilized with the two-part tunneling device 1400. The first tunneling device 1404 would be inserted through the first subdermal incision 1506 and follow the first tunneling device path 1508 towards the mid axillary line 1502 below the armpit 1504 of the patient. When the first tunneling device 1404 reaches the mid axillary line 1502 below the armpit 1504, a second subdermal incision 1512 is performed. The first end 1410 of the first tunneling device 1404 is pulled through the second subdermal incision 1512. The first end 1406 of the second tunneling device 1402 is then inserted through the second subdermal incision 1512 and guided towards the bottom of the rib cage towards the paralumbar line along the second tunneling device path 1510. When the first end 1406 of the second tunneling device 1402 reaches the vertebral body and a medial lateral incision is performed, the second end 1408 of the second tunneling device 1402 is then optionally coupled to the first end 1410 of the first tunneling device 1404 forming the two-part tunneling device. The catheter tube is then threaded through the two-part tunneling device exiting through the first end 1406 of the second tunneling device 1402 through the medial lateral incision. The combined two-part tunneling device is then retracted over the catheter tube and extracted from the first subdermal incision 1506.

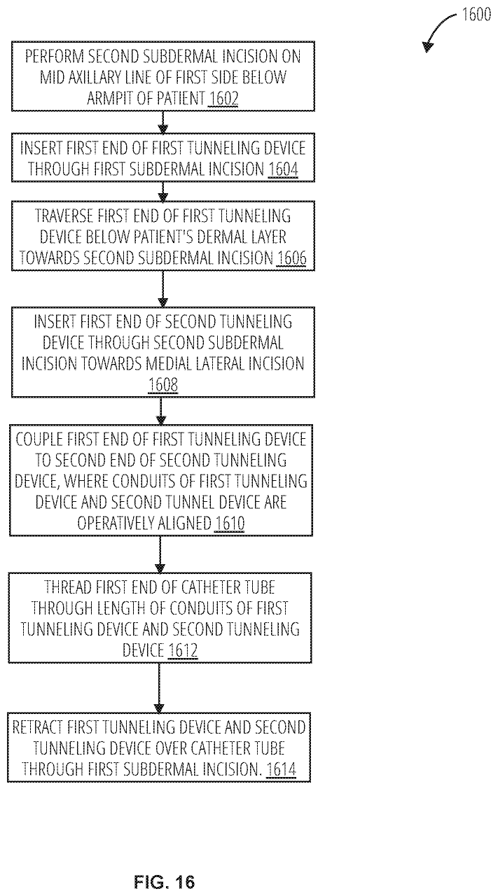

Referencing FIG. 16, a method 1600 for installing an implantable nerve block intervention device utilizing a two-part tunneling device in a patient involves performing a second subdermal incision on the mid axillary line of the first side below the armpit of the patient (block 1602). In block 1604 of method 1600, a first end of a first tunneling device is inserted through the first subdermal incision. In block 1606 of method 1600, first end of the first tunneling device traverses below the patient's dermal layer towards the second subdermal incision. In block 1606 of method 1600, a first end of a second tunneling device is inserted through the second subdermal incision towards the medial lateral incision. In block 1608 of method 1600, the first end of the first tunneling device is coupled to a second end of the second tunneling device, where the conduits of the first tunneling device and the second tunneling device are operatively aligned. In block 1610 of method 1600 the first end of the catheter tube is threaded through the length of the conduits of the first tunneling device and the second tunneling device. In block 1612 of method 1600, the first tunneling device and the second tunneling device are retracted over the catheter tube through the first subdermal incision.

1-6. (canceled)

7. A method for implanting an implantable nerve blocking catheter device comprising:
performing a first subdermal incision on a first side of a ventral lateral portion of a patient's thorax, wherein the first subdermal incision is located between the patient's collar bone and nipple, where the first subdermal incision is found superior lateral to the nipple or in alternate locations;
inserting a first end of a tunneling device, comprising a conduit, through the first subdermal incision;
traversing the first end of the tunneling device below the patient's dermal layer towards a mid-axillary line of the patient on the first side, where the tunneling device path is below the patient's armpit;
traversing the first end of the tunneling device above a lower section of the patient's rib cage towards the patient's paralumbar line from the mid axillary line, proximal to a vertebral body;
performing a medial lateral incision adjacent and slightly lateral to the vertebral body and the first end of the tunneling device;
inserting a tip of a needle or trocar, through the medial lateral incision towards a cluster of nerves, where the needle tip is disposed towards the cluster of nerves;
threading a distal end of the catheter through the needle or trocar, wherein the tip is proximate to the cluster of nerves;
removing the needle or trocar with the catheter left in place;
anchoring the catheter;
threading a proximal end of the catheter back through the tunneling device to a most proximal incision or the first subdermal incision,
retracting the tunneling device over the catheter tube through the first subdermal incision; and
coupling the proximal end of the catheter tube, opposite the distal end of the catheter tube, to a percutaneous access port.

8. The method for implanting an implantable nerve blocking catheter device of claim 7 further comprising:
closing the distal end of the catheter tube and the needle or trocar beneath the medial lateral incision, installing the percutaneous access port through the first subdermal incision, and closing the first subdermal incision, in response to observed dye dispersal over the cluster of nerves from a catheter tip of the implanted nerve blocking catheter device following injection of a dye through the percutaneous access port.

9. The method for implanting an implantable nerve blocking catheter device of claim 7 further comprising:
performing the medial lateral incision adjacent to the patient's vertebral body of lumbar one (L1).

10. The method for implanting an implantable nerve blocking catheter device of claim 9 further comprising:
inserting the needle or trocar tip through the longitudinal paramidline lateral incision towards the patient's celiac plexus in the prevertebral space of the L1, through the parietal peritoneum, threading a catheter through it to the needle tip before withdrawing the needle or trocar and anchoring the catheter.

11. The method for implanting an implantable nerve blocking catheter device of claim 9 further comprising:
inserting the needle or trocar tip through the medial lateral incision towards the patient's lumber sympathetic chain in a vertebral lateral aspect of L1 with subsequent catheter placement and anchoring of the catheter prior to needle or trocar removal.

12. The method for implanting an implantable nerve blocking catheter device of claim 7 further comprising:
performing a second subdermal incision on the mid-axillary line of the patient on the first side below the patient's armpit;
inserting a first end of a first tunneling device through the first subdermal incision; traversing first end of the first tunneling device below the patient's dermal layer towards the second subdermal incision;
inserting a first end of a second tunneling device through the second subdermal incision towards the medial lateral incision;
coupling the first end of the first tunneling device to a second end of the second tunneling device, where the conduits of the first tunneling device and the second tunneling device are operatively aligned;
threading the distal end of the catheter tube through the length of the conduits of the first tunneling device and the second tunneling device; and
retracting the first tunneling device and the second tunneling device over the catheter tube through the first subdermal incision.

13. An implantable nerve blocking device comprising:
a catheter tube, wherein the catheter tube comprises a proximal end and a distal end;
a catheter tip;
an anchoring device; and
a percutaneous access port.

14. The implantable nerve blocking device of claim 13, wherein the anchoring device comprises a non-crimping anchor.

15. The implantable nerve blocking device of claim 13, wherein the anchoring device is configured to anchor the distal end in a prevertebral location.

16. The implantable nerve blocking device of claim 13, wherein the anchoring device is configured to anchor the distal end in a paravertebral location.

17. The implantable nerve blocking device of claim 13, wherein the catheter tube is in fluid communication with the percutaneous access port.

18. A method for using an implantable nerve blocking catheter device comprising:
injecting an anesthetic, other injectate, or combinations thereof through a percutaneous access port of the nerve blocking catheter device;
allowing the anesthetic, other injectate, or combinations thereof to flow from the percutaneous access port though a length of tubing of the nerve blocking catheter device;
allowing the anesthetic, other injectate, or combinations thereof to exit the nerve blocking catheter device via a distal catheter tip anchored proximate to a location of an autonomic or visceral nerve structure, wherein the anesthetic, other injectate, or combinations thereof contacts the autonomic or visceral nerve structure.

19. The method of using an implantable nerve blocking catheter device of claim 18 further comprising:
injecting a second anesthetic, other second injectate, or combinations thereof through the percutaneous access port of the nerve blocking catheter device;
allowing the second anesthetic, other second injectate, or combinations thereof to flow from the percutaneous access port though the length of tubing of the nerve blocking catheter device;
allowing the second anesthetic, other second injectate, or combinations thereof to exit the nerve blocking catheter device via the distal catheter tip anchored proximate to the location of an autonomic or visceral nerve structure, wherein the second anesthetic, other second injectate, or combinations thereof contacts the autonomic or visceral nerve structure.

20. The method of using an implantable nerve blocking catheter device of claim 18, wherein the distal catheter tip is anchored in a prevertebral space of L1 adjacent to the celiac plexus.

21. The method of using an implantable nerve blocking catheter device of claim 19, wherein the distal catheter tip is anchored in a prevertebral space of L1 adjacent to the celiac plexus.

22. The method of using an implantable nerve blocking catheter device of claim 18, wherein the distal catheter tip is anchored in a prevertebral space of L5 adjacent to the superior hypogastric plexus.

23. The method of using an implantable nerve blocking catheter device of claim 19, wherein the distal catheter tip is anchored in a prevertebral space of L5 adjacent to the superior hypogastric plexus.

24. The method of using an implantable nerve blocking catheter device of claim 18, wherein the distal catheter tip is anchored in a paravertebral space of L2, L3, or L4 adjacent to lumbar sympathetic nerves.

25. The method of using an implantable nerve blocking catheter device of claim 19, wherein the distal catheter tip is anchored in a paravertebral space of L2, L3, or L4 adjacent to lumbar sympathetic nerves.

26. The method of using an implantable nerve blocking catheter device of claim 18, wherein the anesthetic, other injectate, or combinations thereof contacts the autonomic or visceral nerve structure to treat chronic abdominal pain, loin pain hematuria syndrome, chronic pelvic syndrome, complex regional pain syndrome associated with the activity of the lumbar sympathetic chain, phantom limb pain, reflex sympathetic dystrophy, causalgia, pain secondary to frostbite, atherosclerosis, Beurger's disease, arteritis secondary to collagen vascular disease; acute pancreatitis, chronic pancreatitis, or combinations thereof.
